(19) United States
(12) Patent Application Publication
(10) Pub. No.: US 2023/0183777 A1
(43) Pub. Date: Jun. 15, 2023

(54) HYPOOSMOTIC STABILIZER FOR GENOMIC DNA OF GUT MICROBIOTA, AND PREPARATION METHOD AND USE THEREOF

(71) Applicant: BioMed Technology Holdings Limited, Hong Kong (CN)

(72) Inventors: Junwei ZHOU, Hong Kong (HK); King Fan Steven LOO, Hong Kong (HK)

(21) Appl. No.: 17/972,906

(22) Filed: Oct. 25, 2022

(30) Foreign Application Priority Data
Dec. 13, 2021 (HK) 32021044409.9

(51) Int. Cl. C12Q 1/6806

(52) U.S. Cl. CPC C12Q 1/6806

(57) ABSTRACT

The present invention provides a hypoosmotic stabilizer for genomic DNA of gut microbiota, comprising: 0.2% Tween 20, 0.1×PBS, and 100-200 mM guanidine thiocyanate. The hypoosmotic stabilizer for genomic DNA of gut microbiota has a pH between 7.0 and 7.4. The hypoosmotic stabilizer for genomic DNA of gut, microbiota as described in the present invention allows a gut microbiota sample to be conveniently and safely stored for 3 weeks (at room temperature) without using a refrigerator for a low storage temperature, and for even longer at a lower temperature. Moreover, the agent is non-toxic, easy-to-use, cheap, and easier to be widely used. The invention further provides a preparation method and use of the hypoosmotic stabilizer for genomic DNA of gut microbiota.

### TECHNICAL FIELD

The invention belongs to the field of biochemistry and molecular biology, in particular to a chemical agent for protecting the stability of genomic DNA of human gut microbiota.

### BACKGROUND

There are a large number of meaningful microorganisms in human and animal guts, which are closely related to the health of the host, and have received extensive attention from academia and industry around the world. In recent years, many research institutions and enterprises have devoted themselves to the study of the relationship between gut microbiota and human diseases, as well as the service of gut health testing. At present, gut microbiota testing has been widely carried out in the US, and is rapidly developed in China although still at an early stage. The key to the gut health assessment and the gut microbiota transplant donor health screening is the ability to extract high-quality genomic DNA of gut microbiota. The genomic DNA of gut microbiota is, easily degraded at room temperature due to the presence of a large amount of nuclease in gut. Therefore, maintaining the stability of the genomic DNA of gut microbiota has become an important prerequisite for the effective extraction of the genomic DNA of gut microbiota. Stabilizers for genomic DNA of gut microbiota can effectively protect the integrity of the genomic DNA of gut microbiota during the transportation and operation of a sample, and are also critical for the ecological analysis and the metabolic characteristics testing of gut microbiota. Especially for samples that need to be shipped at room temperature, the difficulty of temperature control during transportation will result in serious DNA degradation, while low temperature and constant temperature will greatly increase the costs, so they are more dependent on an economical and effective stabilizer for genomic DNA of gut, microbiota.

Formaldehyde has been widely used in stabilizers for genomic DNA, which can stabilize cell membranes to reduce the cell lysis, and can also inhibit nuclease to inhibit the enzymatic reactions, e.g., the DNA degradation caused by DNases. However, the use of formaldehyde or formaldehyde-releasing substances has obvious disadvantages: they may reduce the separation efficiency of nucleic acids by inducing a cross-linking between nucleic acid molecules or between a protein molecule and a nucleic acid molecule. At present, although some progress has been made in the research and development of stabilizers for genomic DNA, new demands keep springing up: especially in the emerging field of gut microbiota testing, maintaining stability of microbial DNA at room temperature for a long term is critical to improve the sensitivity and accuracy of testing and control the transportation costs.

Under the above background, the invention develops a novel hypoosmotic stabilizer for genomic DNA of gut microbiota, which effectively solves the problem that the genomic DNA of human gut microbiota is prone to degradation at room temperature, realizes the long-term preservation of the genomic DNA of gut microbiota at room temperature and further improves its stability.

### SUMMARY

An objective of the present invention is to provide a method capable of stabilizing genomic DNA of gut microbiota, which can address the disadvantages of the existing stabilizer technology for genomic DNA of gut microbiota, protect the stability of all microbial genomic DNA in a sample as comprehensively as possible, and achieve that: 1) the working conditions of such protection is adapted to room temperature; and 2) the effective time of the protection is extended to three weeks, so as to ensure that the samples are adapted to common transportation conditions (e.g., shipping).

To achieve the above object, the present invention utilizes the following technical scheme:

| | |
|---|---|
| Tween 20 | 0.2% |
| Phosphate buffered saline (PBS) | 0.1× |
| Guanidine thiocyanate | 100-200 mM |

The hypoosmotic stabilizer for genomic DNA of gut microbiota as described in the invention is a colorless non-toxic and transparent solution, which can rapidly diffuse into the cytoplasm of gut microbiota. The components at a specific ratio can rapidly and effectively inactivate the DNases in the gut samples so that the DNAs in the samples will not be degraded at room temperature within a certain period of time. This agent can avoid the inconvenience of using a constant temperature or low temperature, and can also effectively solve the problem of transportation of gut microbial samples at room temperature. The beneficial effects of the stabilizer for DNA of the invention are embodied in the following aspects:

Human gut microbiotas are numerous (3-10 times of the total number of human cells) and diverse (more than 1000 types), and thus various DNases from bacteria are abundant and can lead to rapid degradation of DNAs. The stabilizers for DNA of the invention comprises a plurality of different effective components. Guanidine thiocyanate is an uncoupling agent, which is a powerful protein denaturant and can rapidly inactivate DNases to protect the DNAs from degradation by DNases. Tween 20, another component in the stabilizer for DNA of the invention, is a nonionic surfactant, which can, enhance the permeability of cell membrane to accelerate guanidine thiocyanate to enter microbial cells to play the role of protecting the genomic DNAs. The hypoosmotic stabilizer for genomic DNA of gut microbiota of the invention is a non-specific protein denaturant. It can not only inhibit and inactivate the DNases to protect the DNAs, but also inhibit and denature most of other proteases so that it can further protect the integrity of RNAs, expression profiles and proteins in samples. The 0.1×PBS in the stabilizer for DNA of the invention is a hypoosmotic buffer (1×PBS is an iso-osmotic buffer), which can make microbial cells in an expansion state, thereby greatly improving the action efficiency of Tween 20 and guanidine thiocyanate. The stabilizer for genomic DNA stabilizer of the invention can be stored at 4-25° C., which is very suitable for the preservation of human gut microbiota samples.

The invention further provides a method of preparing the hypoosmotic stabilizer for genomic DNA of gut microbiota comprising the steps of:

adding guanidine thiocyanate into water, placing the mixture into a water bath at 80° C. to completely dissolve the guanidine thiocyanate, followed by adding a phosphate buffered saline (1×PBS) and an aqueous solution of Tween 20 in sequence, mixing well and then adjusting pH to 7.0-7.4, and diluting to volume with ultrapure water such that the required concentration of each component is reached, thereby obtaining the hypoosmotic stabilizer for genomic DNA of gut microbiota of the invention.

In the method of preparing the hypoosmotic stabilizer for genomic DNA of gut microbiota as described in the present invention, the water is preferably ultrapure water.

In the method of preparing the hypoosmotic stabilizer for genomic DNA of gut microbiota as described in the present invention, the pH is preferably adjusted with NaOH.

### BRIEF DESCRIPTION OF DRAWINGS

FIG. 1 shows comparison of protective effects in samples (the diversity of microbiotas in the samples) preserved with the inventive hypoosmotic stabilizer for genomic DNA of gut microbiota, a PBS buffer and a commercially available product, respectively, at room temperature for 1 week.

FIG. 2 shows comparison of protective effects in samples (the diversity of microbiotas in the samples) preserved with the inventive hypoosmotic stabilizer for genomic DNA of gut microbiota, a PBS buffer and a commercially available product, respectively, at room temperature for 2 weeks.

FIG. 3 shows comparison of protective effects in samples (the diversity of microbiotas in the samples) preserved with the inventive hypoosmotic stabilizer for genomic DNA of gut microbiota, a PBS buffer and a commercially available product, respectively, at room temperature for 3 weeks.

### DETAILED DESCRIPTION

In the technical schemes of the present invention, all of the ingredients or components, such as, guanidine thiocyanate, phosphate buffered saline (PBS), Tween 20, ultra-pure water, etc., are existing chemicals, or chemicals which can be prepared from existing products by existing methods. The sources of various chemicals are shown in Table 1 below:

| Drug Name | Manufacturer | Code |
|---|---|---|
| Tween 20 | Sigma | 9005-64-5 |
| Phosphate buffered saline | Sigma | p4417 |
| (PBS) | | |
| Guanidine thiocyanate | Sigma | 593-84-0 |

In the preparation method of the invention, the methods of preparing the phosphate buffered saline (PBS) and the aqueous solution of Tween 20 are as follows:

Formulation of a phosphate buffered saline (1×PBS): One phosphate buffered saline (PBS) tablet was dissolved in 800 ml ultrapure water, diluted to 1000 ml, and stored at 4° C.

Formulation of a 5% aqueous solution of Tween 20: 2.5 ml of Tween 20 was fully dissolved in 40 ml of ultrapure water, diluted to 50 ml, and stored at 4° C.

Hereinafter, the technical schemes of the present invention are further described by examples, and the beneficial effects of the hypoosmotic stabilizer for genomic DNA of gut microbiota of the present invention are demonstrated by relevant experiments.

### Example One

400 ml of ultrapure water was added into a 1 L sterilized glass flask, and then 17.7 g of guanidine thiocyanate was added. The mixture was placed in a water bath at 80° C. for complete dissolution. Subsequently, the agents formulated in according the above methods were added in turn. 100 ml of phosphate buffered saline (PBS) and 40 ml of 5% aqueous solution of Tween 20 were well mixed, adjusted with 5N and 1N NaOH to pH 7.0-7.4, diluted to 1 L with ultrapure water, and stored at 4° C.

The prepared hypoosmotic stabilizer for genomic DNA of gut microbiota comprises: 0.2% Tween 20, 0.1× phosphate buffered saline (PBS), and 150 mM guanidine thiocyanate.

### Application Example One

A hypoosmotic stabilizer for genomic DNA of gut microbiota was prepared with components as described above, and subject to a comparison experiment with a PBS buffer and a commercially available DNA stabilizer. The specific steps are as follows:

1 ml of the hypoosmotic stabilizer for genomic DNA of gut microbiota provided by this example, 1 ml of PBS, and 1 ml of the commercially available DNA stabilizer were each added into a 5 ml tube for the collection of a gut microbiota sample (feces), and the sample size collected in each tube is about 10-100 μl.

The collected samples were placed at room temperature to simulate the temperature of a transportation environment. The genomic DNA of gut microbiota was extracted after the 1st week, the 2nd week, and the 3rd week, and a nucleic acid extracting agent (DNeasy Blood & Tissue Kit) available from QIAGEN HONG KONG PTE. LIMITED was selected as the extraction kit.

The obtained DNA was amplified by 16S gene PCR, and then sequenced in MiSeq (Illumina). By comparing the diversity of microbiotas in samples, the protective effects of the stabilizers on the genomic DNA of gut microbiota can be estimated.

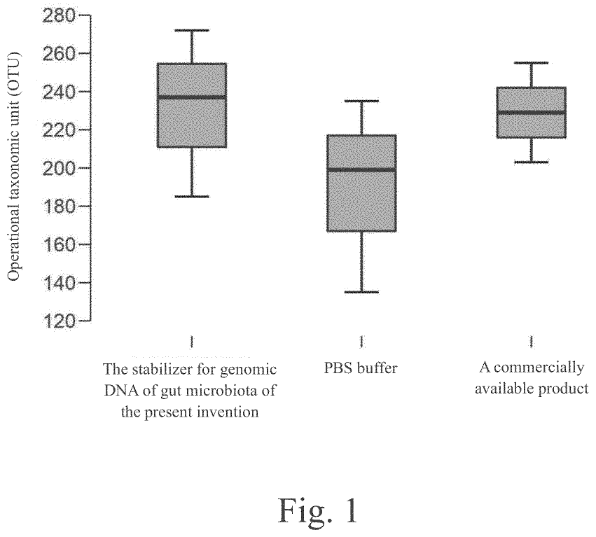

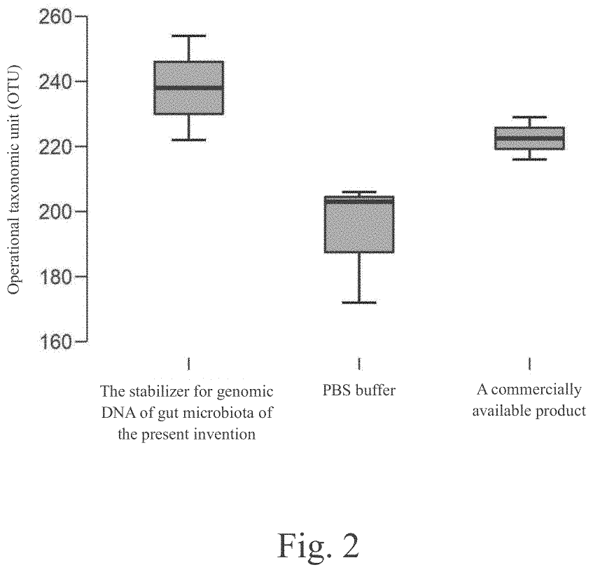

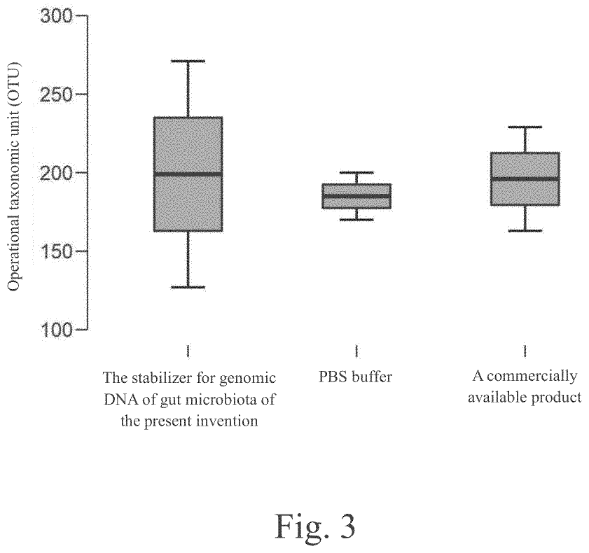

The results are shown in FIGS. 1-3.

As shown in FIGS. 1-3, after adding the hypoosmotic stabilizer for genomic DNA of gut microbiota of the present invention to human gut microbiota samples (feces), the diversity of microbiotas measured by sequencing at three time points (weeks 1, 2 and 3) was significantly higher than, those measured in samples with, the PBS buffer and the commercially available DNA stabilizer (FIGS. 1-3 corresponds to the measurement results of weeks 1-3, respectively). Moreover, the measured diversity of microbiotas did not decrease significantly with the increase of storage time at room temperature. It is indicated that 1) the inventive product (the hypoosmotic stabilizer for genomic DNA of gut microbiota) produces a significant protective effect on the genomic DNA in human gut microbiota samples; and 2) as compared with similar products, the inventive product (the hypoosmotic stabilizer for genomic DNA of gut microbiota) produces a better protective effect on the genomic DNA in human gut microbiota samples.

1. A hypoosmotic stabilizer for genomic DNA of gut microbiota, comprising: 0.2% Tween 20, 0.1×PBS, and 100-200 mM guanidine thiocyanate, wherein the hypoosmotic stabilizer for genomic DNA of gut microbiota has a pH between 7.0 and 7.4.

2. The hypoosmotic stabilizer for genomic DNA of gut microbiota of claim 1, wherein a hypoosmotic phosphate buffered saline (0.1×PBS) is used.

3. The hypoosmotic stabilizer for genomic DNA of gut microbiota of claim 1, wherein the hypoosmotic phosphate buffered saline (0.1×PBS) is used in combination with 100-200 mM guanidine thiocyanate.

4. The hypoosmotic stabilizer for genomic DNA of gut microbiota of claim 1, wherein the hypoosmotic stabilizer for genomic DNA of gut microbiota has a pH between 7.0 and 7.4.

5. A method of preparing the hypoosmotic stabilizer for genomic DNA of gut microbiota of claim 1, comprising the steps of: adding guanidine thiocyanate into water, placing the mixture into a water bath at 80° C. to completely dissolve the guanidine thiocyanate, followed by adding a phosphate buffered saline (1×PBS) and an aqueous solution of Tween 20 in sequence, mixing well and then adjusting pH to 7.0-7.4, and diluting to volume with water such that the required concentration of each component is, reached, thereby obtaining the hypoosmotic stabilizer for genomic DNA of gut microbiota of the invention.

6. The method of claim 5, wherein the water is ultrapure water.

7. The method of claim 5, wherein the pH is adjusted with NaOH.

8. Use of the hypoosmotic stabilizer for genomic DNA of gut microbiota of claim 1 for bacteria preservation, yeast preservation, plant tissue preservation, animal tissue preservation, adherent cell preservation, suspension cell preservation, human and animal skin microbiota, preservation, human and animal oral microbiota preservation, human and animal gut microbiota preservation, and human and animal reproductive tract microbiota preservation.
